(19) United States
(12) Patent Application Publication
(10) Pub. No.: US 2023/0138360 A1
(43) Pub. Date: May 4, 2023

(54) Systems and Methods for Secure Electronic Storage and Access for Genetic Code

(71) Applicant: Samuel B. Reichberg, Hartsdale, NY (US)

(72) Inventor: Samuel B. Reichberg, Hartsdale, NY (US)

(21) Appl. No.: 17/980,175

(22) Filed: Nov. 3, 2022

Related U.S. Application Data
(60) Provisional application No. 63/275,218, filed on Nov. 3, 2021.

(51) Int. Cl. G06F 21/62; G16H 10/60

(52) U.S. Cl. CPC G06F 21/6245; G16H 10/60

(57) ABSTRACT

Systems and methods for secure electronic storage and access for genetic code are provided. The system includes a genetic code storage computer system in communication with a genetic code database and secure access software code executed by the genetic code storage computer system. The system receives genetic code data from a data source, processes the genetic code data to produce secure genetic code data, and stores the secured genetic code data in the genetic code database in communication with the genetic code storage computer system. The system allows for informed ownership by individuals of their genetic sequence information while securely satisfying the access needs of other individuals/entities to such information, preventing unauthorized access to such information, and allowing for continuous enhancement of the genetic information.

### RELATED APPLICATIONS

The present claims the priority of U.S. Provisional Application Ser. No. 63/275,218 filed on Nov. 3, 2021, the entire disclosure of which is expressly incorporated herein by reference.

### FIELD OF THE INVENTION

The present invention relates to computer-based systems and methods for secure data storage and access. More specifically, the present invention relates to systems and methods for secure electronic storage and access for genetic code.

### RELATED ART

With rare exceptions, every cell in the human body has two different copies of the genome, one from each parent. In addition, the mitochondria, a small cell organelle, contains copies of genomic information inherited from the mother. Chemically (except for some viruses), the genome is made up of deoxy-ribonucleic acid (DNA), which consists of long chains of four chemically different electrically charged (basic) subunits (“nucleotides” or “bases”) with names abbreviated as A, T, C, and G, chemically linked to each other. The order or sequence of these nucleotides constitutes a code from which life arises, is maintained throughout the existence of live organisms or viruses, and is inherited by the progeny.

Rapid scientific advances in the last 70 years have elucidated this code and the mechanisms by which it is translated into features of life, including unique individual and familial inheritable characteristics and disease risk. Accelerating technical developments are making it possible to determine the sequence of nucleic acid in multiple organisms and in the totality or subsections of the 3 billion bases of the two copies of the human genome with increasing efficiency so that their coded information can be used in a variety of fields, including the prognosis, diagnosis, and treatment of disease. This process of discovery is accelerating and will continue to develop in the coming years. Thus, roles of nucleic acid sequences that are yet not understood will increasingly be uncovered in the future.

The composition of these molecules remains largely constant from birth to death, is universally considered confidential and is expected to increase in usefulness through the person's or being's life, and beyond. Healthcare, research, and commercial genomic companies are rapidly generating increasing the amount of genomic data, and technologies are being developed to allow individuals and their healthcare providers secure retrieval of targeted subsets of genomic data and access to new clinical and scientific interpretive information about their private nucleic acid sequences as it becomes available. But the analytical technology is progressing much faster than the ability to understand the genetic code embedded in these sequences, their practical personal, scientific and societal uses, and the abuses it could engender, including discrimination, insurance denials, or worse. Yet, its information remains pertinent throughout the individual's life (that in humans could exceed 100 years) and to their family and surviving progeny. For these reasons, and because improving technology will be used to amend and improve the sequence information itself, these data need to be kept confidential, yet readily accessible by the individuals from whom they have been obtained, their healthcare providers and the authorized scientists that are developing the rapidly evolving understanding of the genetic code.

However, these data generally remain under the control of the institutions that generate them or in electronic health records. Current computer-based information technology (IT) tools, such as the electronic medical record (EMR), or those belonging to commercial genomic companies, do not provide for: (1) the necessary interconnectivity and confidentiality settings that exceed those mandated by the U.S. by laws, such as HIPAA which currently do not provide commensurate confidentiality protection; (2) reliable access through the individual's life or for extended times by the individual, her/his healthcare providers and properly vetted researchers; (3) frictionless, targeted, and authorized secure inbound and outbound access; (4) the ability to edit and add to the sequence information as sequencing technology improves; (5) the ability to make available to the users the growing knowledge of how their specific genetic sequences translate into normal and pathologic features of their body and mind, (6) the ability to repeatedly interrogate short sections of the whole genome replacing current individual molecular tests (as the cost of sequencing long segments of nucleic acid, a process called “Next Generation Sequencing,” decreases, extended genomic sequences are bound to replace current independent individual, often expensive, clinical “tests” with merely the interrogation of smaller sections of the full genome sequence in storage, avoiding the need to obtain a new patient sample, extracting, cloning and detecting specific nucleic acids sequences each time such clinical tests are needed for diagnostic or disease management purposes), (7) proper informed consent by the individual, or by the owners of non-human beings, from whom the sample was obtained, for use in non-profit or profitable research. (Currently often researchers access these sequence data without awareness or consent by the individual, or the owner of the non-human being, to whom it belongs), (8) the ability of the source human individual, non-human owner or surviving family inheritors to safely remove this sequence information from storage fully or partially at will for destruction or any other purpose. Accordingly, the systems and methods disclosed herein address the foregoing, and other, needs.

### SUMMARY

The present disclosure relates to systems and methods for secure electronic storage and access for genetic code. The systems and methods disclosed herein provide novel IT, legal, and other protections, processes, and procedures to allow long-term safe electronic storage and retrieval of individual genetic nucleic acid code and associated information while fully preserving the exercise of property rights over the code by the person to whom it belongs (or the entity/ies that own the animal, plant, or other organism), and providing the means for focused, safe, and frictionless access to the sequence, as explicitly approved by the properly informed owner. The system also includes curating tools and resources to accommodate future extension of sequence and associated information, to continuously update and amend the sequence and associated demographic and clinical information, and to provide and permanently update sequence-related annotations that link health or other effects to the individual's own sequence following scientific, medical and technical advances in the field.

### BRIEF DESCRIPTION OF THE DRAWINGS

The foregoing features of the invention will be apparent from the following Detailed Description of the Invention, taken in connection with the accompanying drawings, in which:

FIGS. 1-2 are diagrams illustrating the system of the present disclosure; and

FIGS. 3-4 are flowcharts illustrating processing steps carried out by the system of the present disclosure.

### DETAILED DESCRIPTION

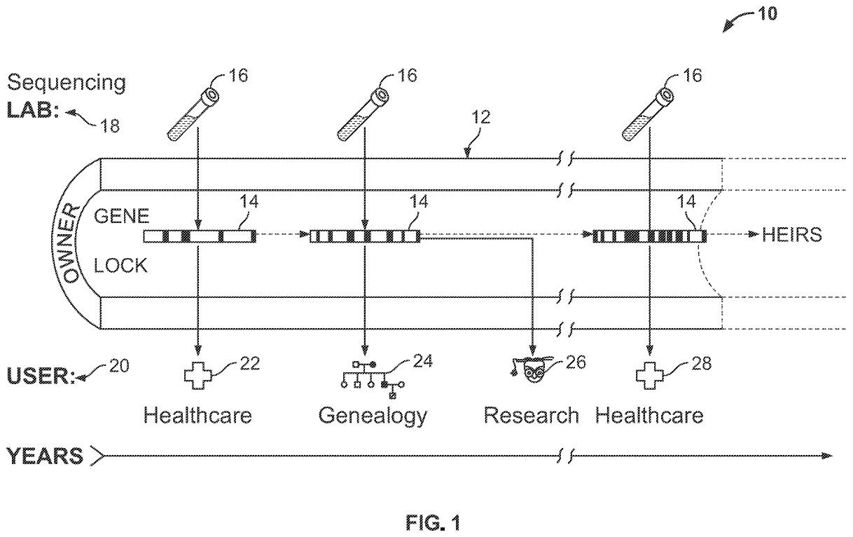

The present disclosure relates to systems and methods for secure electronic storage and access for genetic code, as described in detail below in connection with FIGS. 1-4. As used herein, the term “genetic code” is intended to include not only genetic code such as genome information, DNA, RNA, nucleotides, bases, etc., but also any other genetic data (e.g., individual/familial characteristics, etc.) relating to a human, organism, virus, or other living entity.

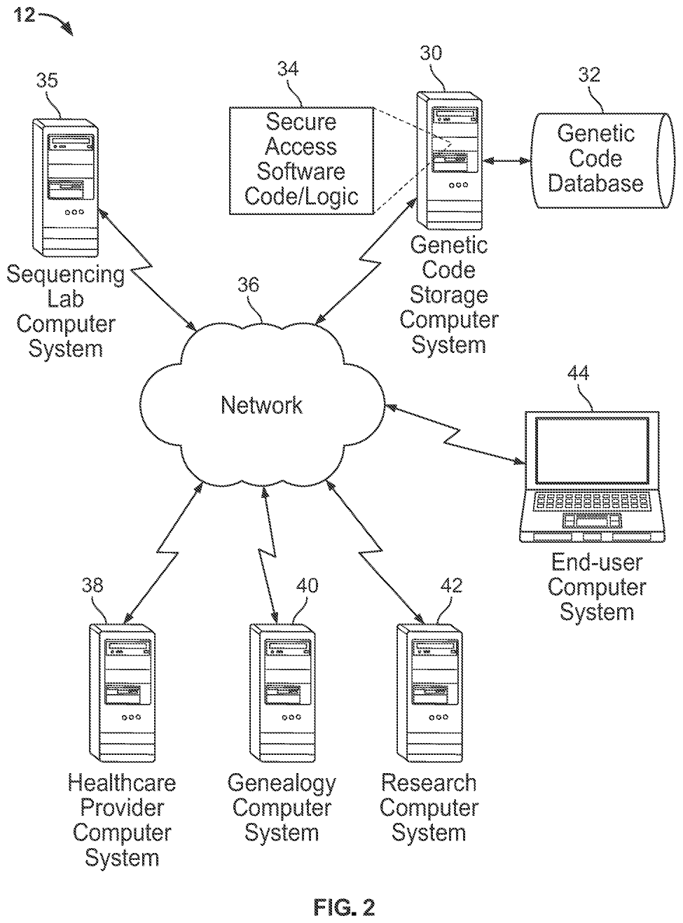

Turning to the drawings, the system 10 of the present disclosure is illustrated in FIGS. 1-2. As shown in FIG. 1, the system 10 provides a computer-based, secure electronic genetic code storage and access platform 12 that securely stores and regulates access to (and dynamic updating of) genetic code 14. The genetic code 14 comprises genetic data that could correspond to one or more laboratory tests 16 conducted by one or more genetic sequencing laboratories 18, and which is securely electronically transmitted to the platform 12 by such laboratories 18. Of course, the code 14 could be transmitted to the platform 12 by other sources, such as third-party data sources, healthcare providers, and other users of the system (e.g., directly from the individuals from whom the genetic code is obtained, and to which the genetic code corresponds).

Electronic access to the code 14 by one or more users 20, such as healthcare providers 24, 28, genealogy service providers 24, and/or educational/research institutions 26, is electronically provided for and controlled by the platform 20. Importantly, and as illustrated by FIG. 1, the system 10 allows for tight control and regulation of the genetic code 14 over a number of years, and most especially, by the individuals who own the genetic code 14 (e.g., the patients/individuals to whom the genetic code 14 corresponds).

FIG. 2 is a diagram illustrating the platform 12 in greater detail. The platform 12 includes one or more secure genetic code storage computer systems 30, which could comprise one or more processors such as a server, a cloud-based computing platform, a standalone computer system, or any other suitable computer system, and which communicate with a secure genetic code database 32 and execute computer-readable secure access software code/logic 34 that, when executed by the computer system(s) 30, cause the computer system(s) 30 to provide the functions and features described herein. As will be discussed in greater detail below, the code 34 allows for secure storage of, and secure access to, genetic code into the database 32, as well as authentication of users who wish to access such genetic code from the database 32. The code 34 could be programmed in any suitable high- or low-level programming language including, but not limited to, C, C++, C#, Java, Python, or any other suitable programming language.

The computer system 30 communicates with one or more sequencing lab computer systems 34 over a network connection 36, which could include, but is not limited to, a local area network (LAN), a wide area network (WAN), the Internet, a wireless communications network, a cellular communications network, or any other suitable communications network. The sequencing lab computer system 34 provides the genetic code to be stored in the database 32, which could be encrypted or otherwise secured before transmission over the network 36 to the computer system 30. Of course, the genetic code could come from another source, as noted above. When the genetic code is received by the computer system 30, it is secured by the system through encryption or other secure processing of the genetic code (e.g., using biometric information, public-key encryption, hash values, or any other suitable encryption technique), to produce secured genetic code data that is then stored by the computer system 30 in the genetic code database 32. Advantageously, by encrypting/securing the genetic code information prior to storage in the database 32, security and control of the genetic code information is further assured in the event that the database 32 and/or computer system 30 is corrupted, hacked, or otherwise tampered with.

One or more end-users of the platform 12 can access the secure genetic code from the database 32 (via one or more computer systems in communication with the computer system 30 via the network 36) only upon successful authentication of such users by the computer system 30. Such end-users could include, but are not limited to, healthcare provider computer system(s) 38, genealogy computer system 40, research computer system 42, or other end-user computer system 44. Each of the systems 38-44 could comprise one or more suitable computer systems, such as personal computers, servers, laptop computers, tablet computers, cellular telephones, mobile computing devices, or other suitable computer systems. The end-user computer system 44 could be utilized by an individual to whom DNA data belongs, and which data is securely handled by the systems and methods of the present disclosure.

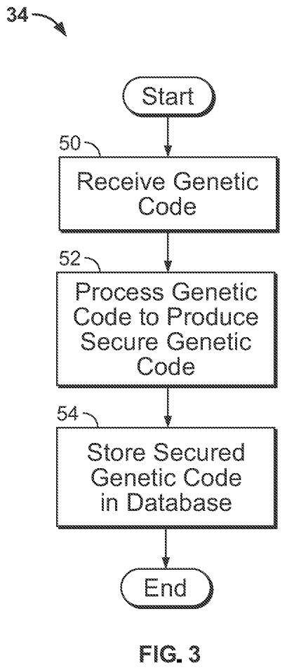

FIG. 3 is a flowchart illustrating processing steps carried out by the software code 34 for securely storing genetic code/data. In step 50, the system 30 receives the genetic code via the network 36, such as from the lab sequencing computer system 34 or some other source. Preferably, the genetic code is securely transmitted to the system 30. In step 52, the system processes the genetic code using a suitable encryption and/or security algorithm (e.g., public-key encryption, RSA encryption, biometric encryption, hash value encryption, or any other suitable encryption/security algorithm) to produce secure genetic code. Such secure genetic code cannot be accessed without knowledge of the particular type of encryption/security algorithm utilized by the system, as well as the relevant access key(s) and/or biometric data needed to access the secured data. Finally, the system 30 stores the secured genetic code in the database 32.

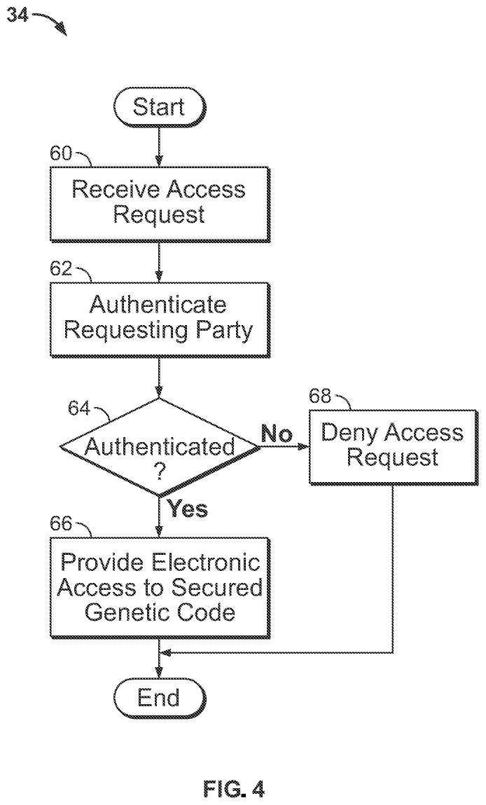

FIG. 4 is a flowchart illustrating further processing steps carried out by the software code 34, for authenticating users and handling incoming requests to access the secure genetic code stored in the database 32. In step 60, the system 30 receives an incoming access request (e.g., transmitted to the system from one or more of the end-user devices 38-44 over the network 36). In step 62, the system 30 authenticates the requesting party (e.g., by user name/password, biometric user identity, or other authentication means). In step 64, the system determines whether the user is authenticated. If so, step 66 occurs, wherein the system 30 provides electronic access to the secured genetic code stored in the database 32. This step includes the process of decrypting the secured genetic code in the database 32, using a decryption algorithm compatible with the encryption algorithm used in step 52 of FIG. 3 and access key(s), biometric data, or other information supplied by the end user of the devices 38-44. Alternatively, if the user is not authenticated, step 68 occurs, wherein the access request is denied by the system 30.

The systems and methods disclosed herein provide several advantages, some of which are discussed in detail below:

Safety: The most secure available barriers, protocols, and contractual means available can be used to keep the information safe from malicious corruption or unauthorized access. Additionally, the systems and methods disclosed herein could implement multiple, concurrent, and/or independent security protocols so as to enhance security and confidentiality of genetic code/data. For example, the system could be configured so as to protect the link between a genetic code and a proxy value assigned to it (which could be a dynamic proxy), and another security system could protect the link between the proxy values and the end users, thus further protecting against attacks by hackers.

Confidentiality: Strict confidentiality of the stored data is observed by restricting access to user's link with the source subject identification and using block chain and similar methods to maintain that link. Restricted access to the sequence data will contribute to confidentiality as it can allow identification of the individual to whom it belongs. Access to the data and the owner's identity will require explicit documented authorization by the owner or her/his representative.

Appending sequence data: An individual will probably accrue partial of complete additional genomic sequence data through her/his life from clinical testing or from parental identification, genealogy, commercial risk assessment, and other services. Improving sequencing technology will also continually be expanding access and accuracy of the sequence obtained from sections of the genome that are difficult to read, such as areas of repeating sequences or pseudogenes. The systems and methods disclosed herein allow for the addition of these data to the individual sequence information already deposited in the system splicing them when the existing data is adjacent.

Amending data: The accuracy of genomic sequence data varies with the technique used, such as the sequencing “depth” (number of analyzed DNA segments that contain the address) or the sequencing method itself. It also varies with the region of the genome being sequenced. For that reason, assignment of one of the four bases to a given address can vary. When adding new data that include segments already in storage, discordances can be vetted and, if necessary, the previously stored data can be amended. All events affecting the stored data, including access, additions or amendments can be audited and documented by the system.

Annotations: Genomic sequences by themselves have little value. They acquire significance when they are linked to biological processes that affect health and to other inheritable characteristics. Narratives of the relationship between a given sequence or single nucleotide and biological features are referred to as “annotations,” which can also be part of the individual sequence information in the system described herein either as copies or links to publicly available information.

Exploration of the genome is a main field of contemporary research, links between specific sequence features and biological features are continuously and copiously reported in scientific and medical literature and catalogued in publicly available data bases. Additionally, the systems/methods herein apply not only to newly-published genomic associations, but also allows for owner notification when such events happen.

Provided below is a hierarchical list illustrating additional processes and/or parameters that can be applied by the system in connection with secure storage and retrieval of sequence data:

Purpose
Healthcare
Investigation
Non-profit
For-profit
Owner notification
Identification
Email
Group
Personal
Bulletin Board
Direct conversation
Secure telephone
Secure text
Read-back
Extent
Access sequence length IT limits
Duration
Access time IT limits - auto removal
Confidentiality
Contractual
Legal
Data transfer IT tools
Owner compensation
Withdrawal fees
Security
Copy/Sharing IT blockade
Modification IT blockade
Auto erasure
Communication of outcome to owner by user

It is further noted that the system can process information related to legal resources, in order to provide additional security and benefits. For example, the system can process and/or follow government confidentiality rules, copyright rules, contractual agreements, research agreements, or other legal resources. Still further, the system can include tools for ensuring permanence of data, such as tools for tracking changes, process changes, and processing other information such as inheritability, insurance information, and organizational information (e.g., corporate information, independence information, commercial interest information, governmental interest information, etc.).

Even further features, benefits, and advantages of the systems and methods of the present disclosure include the following:

### Source and Capture of Sequence and Annotation Data:

A fundamental property of this long-term system for the storage and retrieval of genomic information will be its ability to accommodate on-going scientific and technical advances as well as extension and improvement of the stored information. Importantly, the system can receive information relating to a contractual obligation to preserve the confidentiality of an individual to whom the genetic code data corresponds and defining at least one usage right associated with information contained in the genetic code data, and can maintain the confidentiality of, and enforce usage of, secure genetic code data in accordance with the contractual obligation. Numerous examples of these, and other, contractual obligations which the system and process and enforce are provided below.

Data Content

Nucleic acid sequence information can range from a single nucleotide to the full genome. For many organisms, including humans, the genome has been mapped so that the position of each nucleotide or base is identified with an address. Two of the same or different base pairs (C, G, T or A) belong to each address, one from each parental genome. Mapping of the human genome is relative to a standard map so that only the nucleotides that deviate from the standard genome need to be stored, but each nucleotide address that has been interrogated needs to link to the reference human genome sequence in use at the time sequencing. For the purpose of long-term storage of genomic sequence information, the system described here will have mechanisms to allow the transition to new reference genomes and to new sequence data formats and nomenclature as they become available.

Data Extent

The goal of the system is to store, protect and curate the whole genome of individual subjects, which in humans, consists of 6.4 billion (6.4×10⁹) base pairs. Because of technical, cost, and other limitations, for most individuals this goal will be achieved gradually. Still, due to advances in sequencing technology, it is expected that full exome (the portion of the genome that codes for proteins) or full genome sequencing will become the norm in the near future.

Data Source Clinical Laboratories, genealogy services, parental investigations, commercial health risk assessment services, specialized genome sequencing laboratories, health record, etc.

Subject Identification

Secure identification of the individual whose genomic data is being acquired is paramount and will be carried out using at least four unique identifiers and the most reliable technology available, contractual agreements and, if available, biological identifiers, including previously deposited or externally available genomic identifying information.

Informed Consent

The individual whose genomic information is being deposited into the system, her/his parents, or representatives or, in the case of non-human subjects their owners, will be informed about use of the system utilizing multiple educational means. Their awareness of crucial elements of the conditions will be tested using questions and other educational testing means.

Data Transfer into the System

Transfer of sequence information from sequencing laboratories or other sources will be done using available secure IT methods. Short sequence runs or individual nucleotide site information, will be acquired in a manner that allows future assignment of neighboring nucleotide addresses in a manner analogous to filling a puzzle.

Data Storage

Secure and properly backed-up data storage separate from the Internet will be used for the long-term storage of the subject data. For the purposes of adding to, amending, or retrieving the stored information, a temporary encrypted copy will be made of the sections of interest. This copy will be either securely shared with the requesting party and then deleted or amended and/or appended with the updated information and then securely patched into the permanently stored data.

As electronic storage technology evolves, the data will securely migrate to novel but resilient IT storage containers, maintaining its integrity or, if needed, recoded to maintain their compatibility with evolving genomic data standards.

Safety

The most secure available barriers, protocols, and contractual means available at the time will be used to keep the information safe from malicious corruption or unauthorized access.

Confidentiality

Strict confidentiality of the stored data will be observed by restricting access to their link with the source subject identification and using block chain and similar methods to maintain that link. Restricted access to the sequence data themself will contribute to confidentiality as it can allow identification of the individual to whom it belongs. Access to the data and the owner's identity will require explicit documented authorization by the owner or her/his representative.

Appending Sequence Data

An individual will probably accrue partial or complete additional genomic sequence data through her/his life from clinical testing or from parental identification, genealogy, commercial risk assessment, and other services. Improving sequencing technology will also continually be expanding access and accuracy of the sequence obtained from sections of the genome that are difficult to read, such as areas of repeating sequences or pseudogenes. Methods will be available for the addition of these data to the individual sequence information already deposited in the system, splicing them when the existing data is adjacent.

Amending Sequence Data

The accuracy of genomic sequence information varies with the technique used, such as the sequencing “depth” (number of DNA segments that contain the address which are analyzed) or the sequencing method itself. It also varies with the region of the genome being sequenced. For that reason, assignment of one of the four bases to a given genome address can vary. When adding new data that include segments already in storage, discordances will be vetted and, if necessary, the previously stored data will be amended. All events affecting the stored data, including access, additions or amendments will be audited, documented by the system and conveyed to the owner.

Annotations

Genomic sequences by themselves have little value. They acquire significance when they are linked to biological processes that affect health and other inheritable characteristics. Narratives of the relationship between a given sequence or single nucleotide variant and biological features are referred to as “annotations.” As exploration of the genome is a main field of contemporary research, links between specific sequence features and biological features are continuously and copiously reported in scientific and medical literature and catalogued as annotations in publicly data bases. The system will include connections with these data bases and will attach pertinent annotations to pertinent stored sequences. The owner will be notified and will be provided with links to the corresponding posted annotations.

### Retrieval of Stored Information

Owner Approval

No sequence data or associated information will be made available without explicit and documented approval by the sequence owner or legal representative. The approval will include statements regarding the identity and qualification of the person or institution who originated the request, purpose, extent of the data, duration, confirmation of adherence to confidentiality and safety measures and other agreements.

Purpose

The reasons to examine genomic sequence and related information are numerous and expected to increase rapidly. While maintaining compatibility with various data review interests, the system is optimized for use in the healthcare of the sequence owner and of the species at large through scientific inquiry and research.

Healthcare use of genomic sequence information is nowadays routine in the form of clinical laboratory tests that provide nucleic acid or protein sequence information limited to conditions and diseases of interest. They range from investigations of variations in a single nucleotide (base pair) location to test panels that interrogate variants affecting hundreds or thousands of sites in the genome. They are used to diagnose symptomatic diseases, to screen for diseases of conditions that would otherwise be unnoticed, are a state-of-the art resource in the medical management of cancers, and for other objectives. This information is generally handled the same manner used to report other clinical laboratory results into paper and electronic records of varying formats that are posted to the person or animal electronic or paper-based health record as they are issued. Because of the fragmentation of health records and weak protection of their confidentiality, they provide varying safety barriers and permanence of access to the information. These characteristics are very important in the case of fixed and permanent information that needs to be accessed throughout the subject's life and beyond. With the increased accessibility of genome sequencing, an individual's genomic information will probably end up being obtained once or a few times and followed by repeated access to different sections as required during the subject's life, and that of the family. The system described herein will accommodate this process by providing electronic health record interfaces. Confidentiality of the information will be secured using IT barriers and other tools and by contractual agreements.

Research use of the data will also be managed by the system described here. Access to individual and aggregate genomic data is essential to scientific and medical advances. The system will include tools to make deposited sequences and other genomic information available to research projects that have been approved by appropriate research subject protection institutional boards and are carried out by non-profit or for-profit organizations, but only after well-informed owner documented authorization and system and contractual agreements to security and confidentiality.

Extent of the Retrieved Information

Only the portions of the individual's genomic sequence and related information that are needed for the particular purpose of the inquiry will be made available to the requester. The number of base pairs and location(s) of the requested data will be included in the information provided to the owner for the purpose of approval.

Conditions and Duration of the Retrieved Information

Sequence data and related information retrieved from the system will have limited permanence. According to their use and owner agreement, their availability will be time-limited with durations depending on their purpose, the contractual obligations of the requesting party and the explicit approval by the sequence owner. Derivatives of the information, such as inclusion in analyses that include sequence data from multiple individuals and others, will be allowed to remain available if approved, but the original data will be immutable, will not allowed to be copied or shared and will require complete deletion at the end of the agreed-upon period.

Communications with the Owner

Any change to the deposited and related information will be communicated to the owner through secured and confidential unidirectional or bidirectional means that include receipt confirmation. New and revised sequence annotations will be posted on secure bulletin boards or other means and subjects that share the pertinent features will be notified. Implementations of this system will include educational material and can also include consultation resources. Important and urgent information will be communicated by secure telephone, messaging or similar technology and will include confirmatory read-back or similar practices.

### Termination

The owners of genomic sequences deposited in the system, or their heirs will have the unconditional right to, at any time, remove their information from the system and either have it deleted or transferred to another depository. They will also have the right to copy part, or the totality of their information into another depository.

### Legal

Legal resources will be added to the other means used by the system to protect the deposited information, its confidentiality, and its owner. They include, but are not limited to:
(a) The contractual obligations mentioned in this document
(b) Adherence to Government protective rules and laws, including, but not limited to those governing confidentiality of health information, Civil Rights and Privacy.
(c) A new use of copyright as assigned to the unique genomic sequence of the genome that belongs to an individual. This mode of protection of genetic information is an explicit part of the patent protection requested through this and subsequent applications.

### Costs

The organizational means to implement this system will minimize the cost to the information owners by covering expenses with grants, contracts, service fees and other means.

Having thus described the system and method in detail, it is to be understood that the foregoing description is not intended to limit the spirit or scope thereof. It will be understood that the embodiments of the present disclosure described herein are merely exemplary and that a person skilled in the art can make any variations and modification without departing from the spirit and scope of the disclosure. All such variations and modifications, including those discussed above, are intended to be included within the scope of the disclosure. What is desired to be protected by Letters Patent is set forth in the following claims.

What is claimed is:

1. A system for secure electronic storage and access to genetic code data, comprising:
a genetic code storage computer system in communication with a genetic code database; and
a secure access software code executed by the genetic code storage computer system, the secure access software code causing the computer system to execute the steps of:
receiving genetic code data from a data source;
receiving information relating to a contractual obligation to: (i) preserve confidentiality of an individual to whom the genetic code data corresponds and (ii) defining at least one usage right associated with information contained in the genetic code data;
processing the genetic code data to produce secure genetic code data;
store the secured genetic code data in the genetic code database in communication with the genetic code storage computer system; and
maintaining confidentiality of, and enforcing usage of, the secure genetic code data in accordance with the contractual obligation.

2. The system of claim 1, wherein the genetic code data corresponds to one or more laboratory tests, and the genetic code data is transmitted to the genetic code storage computer system from one or more lab sequencing computer systems in communication with the genetic code storage computer system.

3. The system of claim 1, wherein the step of processing the genetic code data to produce the secure genetic code data comprises encrypting the genetic code data.

4. The system of claim 1, wherein the step of processing the genetic code data to produce the secure genetic code data includes processing the genetic code data using one or more of biometric information, public-key encryption, or a hash value.

5. The system of claim 1, wherein the secure access software code further causes the computer system to execute the steps of:
receiving an access request from an end-user device in communication with the genetic code storage computer system; and
authenticating a party requesting access.

6. The system of claim 5, wherein the secure access software code further causes the computer system to execute the steps of denying the access request if the requesting party is not authenticated or providing electronic access to the secured genetic code from the genetic code database if the requesting party is authenticated.

7. The system of claim 6, wherein the secure access software code further causes the computer system to decrypt the secured genetic code if the requesting party is authenticated.

8. The system of claim 5, wherein the end-user device comprises one or more of a healthcare provider computer system, a genealogy computer system, or a research computer system.

9. The system of claim 1, further comprising receiving enhanced genetic information corresponding to the secure genetic code data.

10. The system of claim 9, further comprising updating the secure genetic code data with the enhanced genetic information.

11. A method for secure electronic storage and access to genetic code data, comprising the steps of:
receiving at a genetic code storage computer system genetic code data from a data source;
receiving information relating to a contractual obligation to: (i) preserve confidentiality of an individual to whom the genetic code data corresponds and (ii) defining at least one usage right associated with information contained in the genetic code data;
processing the genetic code data to produce secure genetic code data;
store the secured genetic code data in a genetic code database in communication with the genetic code storage computer system; and
maintaining confidentiality of, and enforcing usage of, the secure genetic code data in accordance with the contractual obligation.

12. The method of claim 11, wherein the genetic code data corresponds to one or more laboratory tests, and the genetic code data is transmitted to the genetic code storage computer system from one or more lab sequencing computer systems in communication with the genetic code storage computer system.

13. The method of claim 11, wherein the step of processing the genetic code data to produce the secure genetic code data comprises encrypting the genetic code data.

14. The method of claim 11, wherein the step of processing the genetic code data to produce the secure genetic code data includes processing the genetic code data using one or more of biometric information, public-key encryption, or a hash value.

15. The method of claim 11, further comprising the steps of:
receiving an access request from an end-user device in communication with the genetic code storage computer system; and
authenticating a party requesting access.

16. The method of claim 15, further comprising denying the access request if the requesting party is not authenticated or providing electronic access to the secured genetic code from the genetic code database if the requesting party is authenticated.

17. The method of claim 16, further comprising decrypting the secured genetic code if the requesting party is authenticated.

18. The method of claim 14, wherein the end-user device comprises one or more of a healthcare provider computer system, a genealogy computer system, or a research computer system.

19. The method of claim 11, further comprising receiving enhanced genetic information corresponding to the secure genetic code data.

20. The system of claim 19, further comprising updating the secure genetic code data with the enhanced genetic information.
